(19) United States
(12) Patent Application Publication
(10) Pub. No.: US 2026/0199611 A1
(43) Pub. Date: Jul. 16, 2026

(54) MICROSTRUCTURED NOZZLE

(71) Applicant: invoX Belgium NV, Diepenbeek (BE)

(72) Inventors: Jürgen Rawert, Cologne (DE); Frank Bartels, Hattingen (DE)

(21) Appl. No.: 19/138,117

(22) PCT Filed: Dec. 11, 2023
(86) PCT No.: PCT/EP2023/085093

§ 371 Date: Jun. 12, 2025

(30) Foreign Application Priority Data
Dec. 12, 2022 (EP) 22212835.7
Dec. 19, 2022 (EP) 22214714.2

(51) Int. Cl. A61M 11/00; A61M 15/00; B05B 1/26; B05B 15/40

(52) U.S. Cl. CPC A61M 11/003; A61M 11/007; A61M 15/009; B05B 1/26; B05B 15/40

(57) ABSTRACT

The invention provides for a microstructured nozzle (1) for a device (100) for the generation of an inhalable aerosol of a medically active fluid (2), the microstructured nozzle having a main filter (21), an inlet (3) for unfiltered fluid and an outlet (4) for filtered fluid, the inlet and the outlet defining a direction of flow (X) of the fluid from the inlet to the outlet, the nozzle comprising a substantially flat base plate (5) and a cover plate (6) which may be attached thereto; a main filter area (20) comprising the main filter (21); a filtrate outlet area (30) arranged between the main filter and the outlet in the direction of flow; and a fluid distribution area (40) arranged between the inlet and the main filter area in the direction of flow, wherein in the fluid distribution area is disposed a secondary structure (41) which comprises a plurality of pillar-shaped built-in elements (42) extending from the base plate and/or the cover plate transversely to the flow direction. The invention further provides for an inhalation device (100) for inhalation therapy which comprises such microstructured nozzle.

### FIELD OF THE INVENTION

The present invention relates to the field of inhalation devices for medically active fluids. In particular, the invention relates to a microstructured nozzle for a device for the generation of an inhalable aerosol of a medically active fluid as well as to an inhalation device comprising such microstructured nozzle.

### BACKGROUND OF THE INVENTION

Nebulizers or other aerosol generators for liquids have long been known from the art. Amongst others, such devices are used in medical science and therapy. There, they serve as inhalation devices for the application of active ingredients in the form of aerosols, i.e., small liquid droplets embedded in a gas. Such an inhalation device is known, e.g., from document EP 0 627 230 B1. Essential components of this inhalation device are a reservoir in which the liquid that is to be aerosolized is contained; a pumping device for generation of a pressure being sufficiently high for nebulizing the liquid; as well as an atomizing device in the form of a nozzle. By means of the pumping device, the liquid is drawn in a discrete amount, i.e., not continuously, from the reservoir and fed to the nozzle. The pumping device works without propellant and generates pressure mechanically.

In order to achieve a sufficiently homogenous and fine mist of liquid droplets, usually, relatively high pressures such as 10 bar up to about 300 bar or even higher, are necessary. In order to keep the amount of vaporized liquid for each dose acceptably low, the nebulizing nozzle comprises usually one or several channels, each having a cross section only in the order of several μm² (square micrometers), e.g. from 2 μm² to 200 μm². The channels are present in a nozzle body and are often fabricated using micro technological fabrication techniques such as micro etching, micro lithography and the like.

A specific example of a nozzle that has been implemented in a specific nebulizer is disclosed in US 2005/0001076 A1. The disclosed microstructured nozzle consists of a number of channels produced by microstructuring a plate-shaped member. In the nozzle the channels are located between projections which are arranged side by side in rows and project from a base plate. This microstructured base plate is covered with a cover plate. The channels are narrowly defined in terms of shape, cross sectional area and length. The disclosed nozzle contains a zigzag-shaped filter as the primary structure and a secondary structure downstream of the filter.

Due to the plate-shaped layout of the disclosed microstructured nozzle having an inlet slot for the entry of the pressurized fluid it has been proven difficult to provide a stream of pressurized fluid to be atomized evenly over the entire width of the inlet slot, especially in view of the fact that the pressurized liquid to be atomized is provided by a usually circular tubing or other connection to the pump or pressure generator. This results in an uneven distribution of the pressurized liquid within the nozzle which may especially affect the contact of the fluid with a filter element, may it be zigzag-shaped or have another form.

It is thus an object of the present invention to provide for an improved microstructured nozzle which provides for a more even distribution of the pressurized fluid to be atomized within the nozzle structure, specifically with regard to a filter structure provided in such microstructured nozzle. Further objects of the invention will be clear on the basis of the following description of the invention, examples and claims.

### SUMMARY OF THE INVENTION

In a first aspect, the invention relates to a a microstructured nozzle (1) for a device (100) for the generation of an inhalable aerosol of a medically active fluid (2), the microstructured nozzle having a main filter (21), an inlet (3) for unfiltered fluid and an outlet (4) for filtered fluid, the inlet and the outlet defining a direction of flow (X) of the fluid from the inlet to the outlet, the nozzle comprising:
a substantially flat base plate (5) and a cover plate (6) which may be attached thereto;
a main filter area (20) comprising the main filter (21) constructed as the primary structure, with a plurality of main filter projections (22) arranged side by side in at least one row (23), each main filter projection being formed as an integral component of the base plate and projecting therefrom, the main filter projections being spaced from one another by main filter channels (24) which form a path for the fluid through the nozzle from the inlet to the outlet, while the cover plate, if it is attached to the base plate, covers the main filter projections and the main filter channels;
a filtrate outlet area (30) arranged between the main filter and the outlet in the direction of flow; and
a fluid distribution area (40) arranged between the inlet and the main filter area in the direction of flow,
   wherein in the fluid distribution area is disposed a secondary structure (41) which comprises a plurality of pillar-shaped built-in elements (42) extending from the base plate and/or the cover plate transversely to the flow direction.

In a second aspect, the present invention provides an inhalation device for inhalation therapy which comprises a microstructured nozzle according to the first aspect of the invention.

### BRIEF DESCRIPTION OF THE DRAWINGS

FIG. 1 depicts a cross-sectional view of an exemplary inhalation device (100) comprising a microfluidic nozzle (1);

FIG. 2 depicts an embodiment of the base plate (5) of the present microfluidic nozzle (1), viewed from the side which is initially open and which may be subsequently covered with a cover plate (6) (not shown);

FIG. 3 shows an enlarged top-view of a portion of the pillar-shaped built-in elements (42) of secondary structure as provided in a section of fluid distribution area (40);

FIG. 4 shows an enlarged top-view of a section of the main filter (21);

FIGS. 5A and 5B show perspective views of a microstructured nozzle (1) according to the present invention comprising base plate (5) and cover plate (6) attached to each other; and

FIG. 6 shows a perspective view of a section of base plate (5) of microstructured nozzle (1) as shown in top-view in FIG. 2.

### DETAILED DESCRIPTION OF THE INVENTION

The following terms or expressions as used herein should normally be interpreted as outlined in this section, unless defined otherwise by the description or unless the specific context indicates or requires otherwise:

The words ‘comprise’, ‘comprises’ and ‘comprising’ and similar expressions are to be construed in an open and inclusive sense, as ‘including, but not limited to’ in this description and in the claims. In contrast to this, the terms “consist of”, “consists of” and “consisting of” as used herein are so-called closed language meaning that only the mentioned components are present.

The terms ‘a’ or ‘an’ do not exclude a plurality; i.e. the singular forms ‘a’, ‘an’ and ‘the’ should be understood as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms ‘a’, ‘an’ and ‘the’ hence have the same meaning as ‘at least one’ or as ‘one or more’ unless defined otherwise.

The expressions, ‘one embodiment’, ‘an embodiment’, ‘a specific embodiment’ and the like mean that a particular feature, property or characteristic, or a particular group or combination of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

The terms ‘essentially’, ‘about’, ‘approximately’, ‘substantially” and the like in connection with an attribute or value include the exact attribute or the precise value, as well as any attribute or value typically considered to fall within a normal range or variability accepted in the technical field concerned. For example, the term ‘about’ in connection with a value or range of values as used herein shall mean that such value or range of values includes typical deviations from such values of up to +/−5% (abs.), or up to +/−4%, or up to +/−3%, or up to +/−2%, or up to +/−1%, or up to +/−0.5%.

According to the first aspect, the present invention provides for a microstructured nozzle (1) for a device (100) for the generation of an inhalable aerosol of a medically active fluid (2), the microstructured nozzle having a main filter (21), an inlet (3) for unfiltered fluid and an outlet (4) for filtered fluid, the inlet and the outlet defining a direction of flow (X) of the fluid from the inlet to the outlet, the nozzle comprising:
a substantially flat base plate (5) and a cover plate (6) which may be attached thereto;
a main filter area (20) comprising the main filter (21) constructed as the primary structure, with a plurality of main filter projections (22) arranged side by side in at least one row (23), each main filter projection being formed as an integral component of the base plate and projecting therefrom, the main filter projections being spaced from one another by main filter channels (24) which form a path for the fluid through the nozzle from the inlet to the outlet, while the cover plate, if it is attached to the base plate, covers the main filter projections and the main filter channels;
a filtrate outlet area (30) arranged between the main filter and the outlet in the direction of flow; and
a fluid distribution area (40) arranged between the inlet and the main filter area in the direction of flow (for distribution of the unfiltered fluid prior to contact with the main filter),
   wherein in the fluid distribution area is disposed a secondary structure (41) which comprises a plurality of pillar-shaped built-in elements (42) extending from the base plate and/or the cover plate transversely to the flow direction.

The present invention provides for a microstructured nozzle for a device for the generation of an inhalable aerosol of a medically active fluid. Such inhalation devices for the generation of an inhalable aerosol of a medically active fluid, or, in other words, atomizers have been described in the prior art, for example in US 2005/0001076 A1 as discussed above and the references cited therein. Another inhalation device has been disclosed in WO 2018/197730 A1 the contents of which are herewith incorporated by reference in their entirety. These inhalation devices usually have a small size so that they can be held and operated by a user with a single hand. They usually comprise a reservoir for holding the medically active fluid to be aerosolized and administered, a pumping unit for pressurizing the medically active liquid, an actuating mechanism as well as a nozzle unit through which the medically active fluid pressurized by the pumping unit is aerosolized or, in other words, atomized. Such nozzle unit, according to the present invention, may comprise a microstructured nozzle as described in further detail below.

The microstructured nozzle according to the present invention is suitable for the generation of an inhalable aerosol of a medically active fluid, wherein the term “medically active fluid” as used herein refers to a pharmaceutically acceptable liquid compound or composition, particularly to a liquid compound or composition that has pharmacological activity or which comprises a compound or composition which has pharmacological activity and which is capable to improve or prevent symptoms associated with diseases, disorders or conditions, specifically of a disease, disorder or condition of the respiratory system such as pulmonary diseases, disorders or conditions in a subject, specifically in a warm-blooded animal or human, especially in a human. Specific examples of such a disease, disorder or condition comprise, but are not limited to lung diseases or conditions such as asthma and/or chronic obstructive pulmonary disease (COPD), especially COPD, or interstitial lung diseases affecting the interstitium of the lung and lung tissues such as those associated with the air passages and/or air sacs (alveoli), for example pulmonary fibrosis such idiopathic pulmonary fibrosis (IPF), interstitial pneumonias, or sarcoidosis.

Furthermore, the term “inhalable aerosol” refers to an aerosol having respirable particles or droplets, preferably such particles or droplets having a mass median aerodynamic diameter (as measured by laser diffraction) of not more than about 10 μm, in particular of not more than about 7 μm, or of not more than about 5 μm, respectively.

In specific embodiments, the term “medically active fluid” as used herein refers to a medically active fluid or liquid in form of a pharmaceutical composition comprising at least one active pharmaceutical ingredient (API), more specifically at least one inhalable active pharmaceutical ingredient. More specifically, such at least one inhalable active pharmaceutical ingredient may, for example, be selected from long-acting muscarinic antagonists (LAMA), long-acting beta agonists (LABA) and inhalable glucocorticosteroids (ICS), as well as from analgetics and antidiabetics, either alone or in combination which each other.

Examples for long-acting muscarinic antagonists (LAMA) comprise, but are not limited to aclidinium bromide, glycopyrronium salts, such as glycopyrronium bromide, revefenacin, tiotropium, such as tiotropium bromide, umeclidinium bromide, oxitropium bromide, flutropium bromide, ipratropium bromide, trospium chloride, tolterodine.

Examples for long-acting beta agonists (LABA) comprise, but are not limited to, albuterol, arformoterol, bambuterol, bitolterol, broxaterol, carbuterol, clenbuterol, fenoterol, formoterol, hexoprenaline, ibuterol, indacaterol, indacterol, isoetharine, isoprenaline levosalbutamol, mabuterol meluadrine, metaproterenol, olodaterol, orciprenaline, pirbuterol, procaterol, reproterol, rimiterol, ritodrine, salmeterol, salmefamol, soterenot, sulphonterol, tiaramde, terbutaline, terbuterol.

Examples of inhalable glucocorticosteroids (ICS) comprise, but are not limited to, prednisolone, prednisone, butixocort propionate, flunisolide, beclomethasone, triamcinolone, budesonide, fluticasone, mometasone, ciclesonide, rofleponide, dexamethasone, etiprednol-dichloroacetat, deflazacort, etiprednol, loteprednol, RPR-106541, NS-126, ST-26.

Furthermore, active pharmaceutical ingredients may be selected from analgetics, such as opioid analgetics (e.g. morphine, fentanyl) or non-opioid analgetics (e.g. salicylic acid derivates, e.g. acetylsalicylic acid) or cannabinoids (e.g. tetrahydrocannabinol) or antidiabetics, such as insulin.

The medically active fluid that may be nebulized or aerosolized by the present microstructured nozzle may comprise at least one active pharmaceutically ingredient as described above but may also comprise a mixture of two or more active pharmaceutically ingredients that may be administered by inhalation.

The medically active fluid as referred to herein may be in the form of a dispersion, for example a suspension with a liquid continuous phase, and a solid dispersed phase or in the form of a solution, specifically in the form of an aqueous solution. Furthermore, the medically active fluid as referred to herein may comprise, optionally, one or more physiologically acceptable excipients, which are suitable for inhalative use. Excipients which may be featured in the medically active fluid as referred to herein may include, but are not limited to, one or more of buffering agents to regulate or control pH of the solution, salts, taste-masking agents, surfactants, lipids, antioxidants, preservatives and co-solvents, which may be used to enhance or improve solubility, for example water, alcohols, specifically alcohols with 2 to 4, or preferably 2 or 3 carbon atoms, such as ethanol, propanol or iso-propanol or a glycol, such as propylene. In specific embodiments, the medically active liquid as described above may be essentially free or even free of a propellant, such as a hydrofluoroalkane (HFA) propellant.

In specific embodiments, the medically active fluid as referred to herein comprises at least one pharmaceutically active ingredient as described above dissolved in an alcoholic or aqueous liquid vehicle or solvent. In preferred embodiments, such liquid vehicle or solvent comprises water and/or ethanol, preferably ethanol. In further specific embodiments, such liquid vehicle or solvent comprises or preferably consists of ethanol or a mixture of ethanol and water, wherein ethanol may be, for example, comprised in an amount of at least about 50 wt.-%, or at least about 60 wt.-% or at least about 70 wt.-% or even more and water in a corresponding amount of up to about 50 wt.-%, or up to about 40 wt.-% or up to about 30 wt.-% or less. In specific embodiments, the liquid vehicle or solvent comprises or consists of ethanol in an amount of about 60 to about 80 wt.-%, such as about 70 wt.-%, and water in an amount of about 40 to about 20 wt.-%, such as about 30 wt.-%. In further specific embodiments, the liquid vehicle or solvent may comprise or essentially consist or consist of water, for example in an amount of from about 80 wt.-% or 90 wt.-% to about 100 wt.-% (with regard to the total weight of the liquid vehicle), such as from about 85 wt.-% or from about 90 wt.-% or from about 95 wt.-% or even from about 97 wt.-% to about 98 wt.-% or to about 99 wt.-% or to about 99.5 wt.-% and a further solvent or mixture of further solvents, preferably an alcohol or a glycol, specifically ethanol adding to 100 wt.-% of the liquid vehicle.

According to the present invention, the microstructured nozzle has a filter, an inlet for the entry of unfiltered fluid and an outlet for the delivery and atomization of filtered fluid. The inlet and the outlet of the microstructured nozzle define a direction of flow of the fluid from the inlet to the outlet, or, in other words, the direction in which the medically active liquid is delivered in a downstream direction.

The dimensions of the present microstructured nozzle are usually very limited to allow for the implementation in especially atomizers or inhalation devices which are portable and suitable for one-or two-hand operation of a user. In many cases, the microstructured nozzles according to the invention have a miniaturized size with diameters and/or edge lengths of well below 10 mm or even well below 5 mm as described in further detail below. Accordingly, the microstructures provided in the microstructured nozzle as also described in further detail below usually have dimensions one or more orders of magnitude below that.

According to the invention, the microstructured nozzle comprises a substantially flat base plate and a cover plate which may be attached to the base plate. The base plate is preferably structured by etching techniques in a manner known to those of skill in the art as, for example, A A Ayón et al. 2001 Smart Mater. Struct., 10, 1135. In some embodiments, the base plate has a generally square or rectangular plate shape with length of the edges of up to about 5 mm, such as from about 0.5 to about 4 mm or from about 1 mm to about 4 mm or from about 1.5 mm to about 3 mm or from about 2 to 3 mm. In some embodiments, the base plate may have a width (perpendicular to the direction of flow) of from about 1.5 mm or from about 2 mm to about 3 mm, such as about 2.5 mm, and a length (along the direction of flow) of from about 1.5 mm to about 2.5 mm, such as about 2 mm. The height of the base plate usually ranges from about 0.2 mm to about 2 mm, such as from 0.6 mm to about 1.2 mm or from about 0.9 mm to about 1.8 mm or from about 1.2 mm to about 1.5 mm. The heights of the microstructures (of the primary, secondary and tertiary structures) as described in more detail below, in some embodiments, are selected within a range of from about 2 μm to about 40 μm, usually from about 3 μm to about 20 μm, preferably of from about 4 to about 14 microns, and particularly from about 5 μm to about 8 μm or even of from about 5 μm to about 7 μm (as measured from the basis of such microstructures on the base plate). In cases in which the cover plate does not comprise any microstructure or, in other words is provided in the form of a flat cover, this height of the microstructures as referred to above corresponds to the height of the flow channel provided in microstructured nozzle. Due to the manufacturing methods available, in particular embodiments, the height (as measured from the surface of the base plate in the direction of the cover plate) of all microstructures provided on the base plate (including the primary, secondary and tertiary structures as described in detail below), in some embodiments, is the same or substantially the same.

The material that may be used for the base plate is preferably a monocrystalline silicon, as it is cheap and available in a state (i.e. in wafers) in which it is sufficiently flat and parallel with a slight surface roughness, and it can be attached to the cover plate without the additional application of adhesives or other materials during the subsequent connection process. In order to produce a plurality of nozzle arrangements in parallel manner, a plurality of structured base plates may be made from a silicon wafer.

The microstructured nozzle of the invention consists of at least two sheets, preferably of two sheets, preferably of glass and/or silicon, securely fixed together, at least one of which has one or more microstructured channels which connect the nozzle inlet to the nozzle outlet. The nozzle outlet with the outlet openings or, in other words, ejection channels of the microstructured nozzle is preferably on the opposite side from the nozzle inlet. The nozzle inlet may have just one fluid inlet or a plurality of fluid inlets. After passing through the inlet and an optional coarse filter the fluid flows through a filtrate distribution area as described in detail below followed by a main filter formed by a plurality of main filter projections. Behind the main filter, or, in other words, between the main filter and the outlet of the microstructured nozzle viewed in the direction of flow (in “downstream” direction), is a filtrate collecting chamber for fluid which has already been filtered. From the fluid collecting chamber the fluid goes to an outlet which is preferably constructed in the form of a nozzle opening with one or more, preferably two ejection channels.

The present microstructure nozzle further comprises a cover plate corresponding to the second of the at least two sheets. Suitable cover plates may be, for example, sheets of glass such as alkali borosilicate glass, e.g. Pyrex, (Corning) or Tempax (Schott). These may be attached to the base plate, for example, by anodic bonding of the silicon and glass.

In some embodiments, the inlet of the present microstructured nozzle may be located at the inlet end of the base plate and the outlet may be located at the opposite outlet end of the base plate, wherein the inlet and the outlet are connected by opposing sidewalls and wherein the inlet and outlet may define a flow channel through which the medically active fluid flows in the direction of flow (X) or, in other words, in downstream direction. In some embodiments, the outlet comprises at least one ejection channel for the ejection of a jet of the medically active fluid. In other embodiments, the outlet comprises at least two ejection channels adapted to eject at least two jets of medically active fluid. In yet further embodiments, such at least two liquid jets are oriented such that the at least two jets intersect or impinge with each other to form the inhalable aerosol of the medically active fluid. In yet further embodiments, the ejection channel or ejection channels each may have a cross-section with a width of from about 5 μm to about 15 μm or from about 6 μm to about 10 μm, for example, about 8 μm and a height corresponding to the height of the primary, secondary and tertiary microstructures as described in detail below of from about 2 μm to about 40 μm, usually from about 3 μm to about 20 μm, preferably of from about 4 to about 14 microns, and particularly from about 5 μm to about 8 μm or even of from about 5 μm to about 7 μm.

The microstructured nozzle according to the present invention comprises a main filter area comprising a main filter constructed as the primary structure. The main filter is located in the main filter area of the present microstructured nozzle and may be formed on the base plate and/or the cover plate, preferably, however, only on the base plate. In preferred embodiments, the main filter is located and formed on the main filter area of the base plate as described in further detail below. The term “area” as used herein, for example, in connection with the main filter area but also in connection with further areas, such as the filtrate outlet area, the fluid distribution area as well as the first and second interface areas as described in detail below, refers to a particular portion or section of the present microstructured nozzle, specifically to a particular section of the surface of the present microstructured nozzle, more specifically to a particular surface section forming the flow channel of the present microstructured nozzle. More specifically, the term “area” may refer to a section of the surface of the base plate forming or, in other words, facing the flow channel. Even more specifically, the term “area” may refer to a section of the surface of the base plate forming or facing the flow channel of the present microstructured nozzle having essentially the shape of a rectangle or square, preferably an essentially rectangular shape. In some embodiments, such rectangle or square, preferably rectangle section of the flow channel may have a width spanning the entire flow channel from one sidewall to the opposing sidewall and may have a length spanning over a fraction of the total length of the flow channel connecting the inlet of the present microstuctured nozzle with the outlet. The main filter comprises a plurality of main filter projections arranged side by side in at least one row, whereas each main filter projection may be formed as an integral component of the base plate and projects therefrom. Furthermore, the main filter projections are spaced from one another by main filter channels which form a path for the fluid through the nozzle from the inlet to the outlet, while the cover plate, if it is attached to the base plate, covers the main filter projections and the main filter channels.

The projections of the main filter (as well as the main filter area), in some embodiments, extends over the entire width of the flow channel from one opposing sidewall to the other opposing sidewall. The term “width” as used herein in connection with a structure having such width means the extension of such structure in the plane of the flow channel but perpendicular to the direction of flow, e.g., in the case of the present base plate, in the direction spanning from one sidewall to the opposing sidewall perpendicular to the direction of flow. In contrast to this, the term “length” as used herein in connection with a specific structure having such length means the extension of such structure in the plane of the flow channel in the direction of flow, e.g., in the case of the present base plate, in the direction spanning from the inlet to the outlet. Finally, the term “height” as used herein in connection with a specific structure having such height means the extension of such structure perpendicular to the width as well as perpendicular to the length of such structure.

In some embodiments, the main filter area may have a width in the range of up to about 5 mm, such as from about 0.5 to about 4 mm or from about 1 mm to about 4 mm or from about 1.5 mm to about 2.5 mm or from about 2 to 3 mm (perpendicular to the direction of flow) and a length (in the direction of flow) of from about 0.5 mm to about 1.5 mm or from about 0.75 mm to about 1.25 mm. In some embodiments, the main filter area may have a constant width over the entire length of the main filter area or, in other words, may have constant width from the upstream end or inlet of the main filter area to the downstream end or outlet of the main filter area.

In particular embodiments, in the microstructured nozzle according to this aspect of the invention, the main filter comprises a plurality of zigzag projections extending transversely to the flow direction from a base plate, defining a plurality of channels and forming spikes in directions of the inlet and the outlet. In further particular embodiments, the projections of the main filter are arranged side by side over an entire width of the filter or, in other words, from one sidewall of the base plate to the opposing sidewall of the base plate. Accordingly, in some embodiments, the main filter comprises a plurality of zigzag projections extending transversely to the flow direction extends over the entire width of the flow channel from one side wall of the base plate to the opposing sidewall of the base plate.

As described above, the main filter comprises a plurality of projections arranged in rows, preferably in a zigzag shape, projecting from a preferably flat base plate and hence forming an integral part of the base plate. The base plate may be preferably completely covered by a preferably flat cover plate. This forms a plurality of channels between the projections, the base plate and the cover plate. These channels form a passage from the inlet end to the outlet end of the filter nozzle. The spacing between the base plate in the area around the main filter projections and the cover plate within a row of projections is about the same size as the width of the channels on the side of the projections where the fluid enters the series of channels. Unfiltered fluid enters the main filter through the inlet which may be in the form of one or more oblong inlet slot(s). The inlet slot(s) may be about the same height as the projections protruding from the base plate on the inlet side of the filter.

In alternative embodiments, the projections of the main filter may be arranged in several rows in a cascade. The projections arranged closer to the inlet side of the filter may be larger than the projections arranged more on the outlet side of the filter. Again, the spacing between the flat base plate and the flat cover plate in the area around each row of main filter projections arranged in a cascade may be about the same as the width of the channels on the side of the projections where the fluid enters the row of channels. This spacing may be between half and twice the width of the channel. This spacing may decrease from row to row, viewed in the direction of flow. The main filter channels thus may have a substantially square cross section at their entry end for the fluid. In all the embodiments, the spacing between the flat base plate in the area around the projections and the flat cover plate within a row of projections of the main filter may be constant. The spacing may be greater in the region of the end of the row which is close to the outlet side of the main filter than in the region of the end of the row which is close to the inlet side of the filter. This spacing may preferably increase in substantially linear fashion from one end of the row of projections to the other.

In some embodiments, the spacing between neighboring main filter projections and, accordingly the width of the main filter channels may be selected within a range of from about 1 μm to about 25 μm or from about 1.5 μm to about 15 μm or from about 2 μm to about 10 μm. In preferred embodiments, the spacing between neighboring main filter projections is selected from about 2 μm to about 5 μm. In some embodiments, the neighboring main filter elements are equally spaced such that all of the main filter channels have an equal width.

The present microstructured nozzle further comprises a filtrate outlet area arranged between the main filter (or main filter area) and the outlet of the microstructured nozzle in the direction of flow (downstream of the main filter area).

In particular embodiments, the filtrate outlet area is an area formed on the base plate and/or the cover plate, preferably, however, only on the base plate, which comprises a hollow space in which the filtrate (i.e. the filtered medically active fluid) is received after passing the main filter or, in other words, after leaving the main filter area. The filtrate outlet area, in some embodiments, extends over the entire width of the flow channel from one opposing sidewall to the other opposing sidewall and, accordingly, may have a width in the range of up to about 5 mm, such as from about 0.5 to about 4 mm or from about 1 mm to about 4 mm or from about 1.5 mm to about 2.5 mm or from about 2 to 3 mm. The length of the filtrate outlet area (in the direction of flow) may be from about 0.1 mm or from about 0.25 mm to about 1.5 mm or from about 0.1 mm to about 0.6 mm or from about 0.5 mm to about 1.25 mm. While the width of the filtrate outlet area can be varied within broad ranges, e.g. as described above, in further particular embodiments, the filtrate outlet area has (approximately) the same width as the main filter area located upstream of the filtrate outlet area, or, in other embodiments, the same width as the downstream end of the main filter area located upstream of the filtrate outlet area. In further particular embodiments, the upstream end of the filtrate outlet area has (approximately) the same width as the (downstream end of the) main filter area located upstream of the filtrate outlet area.

In further particular embodiments, the filtrate outlet area does not comprise structural elements located in the inner volume (of the hollow space) of the filtrate outlet area such as the primary or secondary structures comprised in the main filter area and the fluid distribution area. In yet further particular embodiments, the filtrate outlet area does not overlap with the main filter area (and, as a consequence does also not overlap with the fluid distribution area located upstream of the main filter area). In other words, in preferred embodiments, the filtrate outlet area is a hollow space for receiving the filtered medically active fluid which, in some embodiments, may have a volume of from about 5 to about 10% of the total inner volume of the microstructured nozzle.

In particular embodiments, the filtrate outlet area may have the same width as the (downstream end) of the main filter area as described above and may gradually or discontinuously narrow in the direction towards the outlet end of the microstructured nozzle, or in further particular embodiments, may open into the at least one outlet channel of the present microstructured nozzle.

The microstructured nozzle according to this aspect of the invention further comprises a fluid distribution area which is arranged (in the direction of flow) between the inlet and the main filter area. The fluid distribution area is especially suitable for the distribution or spreading of the pressurized, unfiltered fluid to be atomized/aerosolized prior to contact with the main filter as described in further detail below.

In particular embodiments, the fluid distribution area is an area formed on the base plate and/the cover plate, preferably, however, only on the base plate, which comprises a hollow space in which a secondary structure as described in further detail below is disposed. The fluid distribution area, in some embodiments, extends over the entire width of the flow channel from one sidewall to the opposing sidewall and, accordingly may have a width in the range of up to about 5 mm, such as from about 0.5 to about 4 mm or from about 1 mm to about 4 mm or from about 1.5 mm to about 2.5 mm or from about 2 to 3 mm. The length of the fluid distribution area (in the direction of flow) may be from about 0.1 mm or from about 0.25 mm to about 1.5 mm or from about 0.1 mm to about 0.6 mm or from about 0.5 mm to about 1.25 mm, preferably from about 0.1 mm to about 0.6 mm or from about 0.2 mm to about 0.5 or from about 0.3 mm to about 0.4 mm. While the width of the fluid distribution area can be varied within broad ranges, e.g. as described above, in further particular embodiments, the fluid distribution area has (approximately) the same width as the main filter area located downstream of the fluid distribution area or, in alternative embodiments, may have the same width as the upstream end of the main filter area. In some embodiments, the fluid distribution area has an essentially constant or uniform width over the entire length of the fluid distribution area. In further particular embodiments, the fluid distribution area has a height (perpendicular to the width and the length as described above) of about 2 μm to about 40 μm, usually from about 3 μm to about 20 μm, preferably of from about 4 to about 14 microns, and particularly from about 5 μm to about 8 μm or even of from about 5 μm to about 7 μm.

In the fluid distribution area of the present microstructured nozzle is disposed a secondary structure which comprises a plurality of pillar-shaped built-in elements extending from the base plate and/or the cover plate, preferably extending from the base plate only, transversely to the flow direction. In order to form the secondary structure in the fluid distribution area additional pillar-shaped built-in elements are constructed. Preferably, these built-in elements of the secondary structure are in the form of cylindrical elevations extending from the bottom of the base plate to the cover plate. They are preferably cylinders of circular cross section. In particular embodiments, all of the pillar-shaped built-in elements of the secondary structure provided in the present microstructured nozzle are disposed in the fluid distribution area. Accordingly, in these embodiments, none of the pillar-shaped built-in elements of the secondary structure are disposed in at least one of the main filter area and the filtrate outlet area, specifically none of the pillar-shaped built-in elements of the secondary structure are disposed in the filtrate outlet area.

In particular embodiments, the height of the built-in elements corresponds to the height of the fluid distribution area as described above. In further embodiments, the built-in elements may be formed out of the base plate or as an integral part of the cover plate. In preferred embodiments, the built-in elements are formed as an integral part of the base plate. In yet further embodiments, all of the main filter projections (of the primary structure) and all of the built-in elements (of the secondary structure) are formed as an integral part on the base plate.

In alternative embodiments, the built-in elements may be formed out of the base plate and out of the cover plate. For example, some of the built-in elements may be formed entirely of the base plate and some of the built-in elements may be formed entirely of the cover plate. In further alternative embodiments, the built-in elements may be partly formed by the base plate and partly formed by the cover plate such that the parts of the corresponding built-in elements combine to the final built-in elements upon coverage of the base plate by the cover plate. In preferred embodiments, the pillar-shaped built-in elements extend from the base plate to the cover plate.

In particular embodiments, the dimensions of the pillar shaped built-in elements of the secondary structure are selected so that they do not substantially increase the flow resistance. This may be achieved by making the spacings between the built-in elements, each of which forms a throughflow channel for the liquid passing through, such that the resulting cross-sectional area perpendicular to the direction of flow which is effectively permeable to the liquid is greater than the corresponding effective cross-sectional area of the throughflow channels formed by the filter structures, more specifically by the primary structure of the main filter. Thus, the flow characteristics of the liquid inside the nozzle are most strongly influenced by the (primary) structures of the main filter.

Accordingly, in preferred embodiments, one or more spacings between the pillar-shaped built-in elements, each of which forms a throughflow channel for the liquid passing through, are such that a resulting cross-sectional area transverse to the direction of flow which is effectively permeable to the liquid is greater than a corresponding effective cross sectional area of the main filter channels formed by the projections of the main filter such that the built-in elements do not substantially increase a flow resistance.

The cross section of the built-in elements is preferably chosen such that the flow resistance for a fluid flowing through is minimized. Round, circular or oval cross sections are preferred for this. As an alternative to the cross sections described above, they may also be triangular, trapezoidal or rectangular, while the angles should be aligned in the direction of flow. In preferred embodiments, however, the built-in elements of the secondary structure of the fluid distribution area have a cylindrical circumferential wall. As an alternative, however, it may also be advantageous to construct the built-in elements with a concave or, alternatively, a convex circumferential wall.

In further preferred embodiments, the dimensions, spacings and the arrangement of the pillar-shaped built-in elements of the secondary structure relative to one another are such that the resulting arrangement of pillar-shaped built-in elements allows for the formation of an interface between the medically active liquid and the surrounding atmosphere, especially on the upstream or downstream end, specifically on the downstream end of the secondary structure in order to allow for the formation of forces resulting from the surface tension of the medically active liquid when in contact with the secondary structure.

In preferred embodiments, the built-in elements of the secondary structure are arranged in parallel rows in an ABAB arrangement with preferably equidistant intervals within rows A and B and between rows A and B. The adjacent rows A and B are preferably displaced in the direction of flow by the diameter of the built-in elements. The use of built-in elements of circular cross section, accordingly, may produce a geometry in which each of the built-in elements forms the center of an equilateral hexagon, each angle being formed by an adjacent built-in element (hexagonal design). In some embodiments, at least some built-in elements form equilateral hexagonal designs, where a center of each of the hexagonal designs is formed by a built-in element and each angle of each of the hexagonal designs is formed by adjacent built-in elements.

Obviously, this only applies to the plurality of built-in elements which are surrounded by an equilateral hexagon and, accordingly, does not apply to built-in elements positioned at the edge or, in other words, adjacent to the upstream or downstream end or the opposing sidewalls of the secondary structure.

In particular embodiments, the pillar-shaped built-in elements of the secondary structure may have a spacing selected within the range from about 5 μm to about 50 μm or from about 5 μm to about 20 μm or in the range of from about 5 μm to about 15 μm, or from about 7.5 μm to about 12.5 μm, such as 10 μm from one another thereby forming the channels of the secondary structure. In further particular embodiments, the pillar-shaped built-in elements of the secondary structure are evenly and regularly distributed over the entire fluid distribution area. In further particular embodiments, the spacing between neighboring pillar-shaped built-in elements is the same over the entire fluid distribution area. In even further embodiments, the plurality of channels of the secondary structure have a constant diameter over the entire height of the secondary structure.

According to further particular embodiments, the pillar-shaped built-in elements have a diameter selected within the from about 5 μm to about 50 μm or from about 5 μm to about 20 μm or in the range of from about 5 μm to about 15 μm, or from about 7.5 μm to about 12.5 μm, such as 10 μm. In preferred embodiments, all pillar-shaped built-in elements of the secondary structure have the same cross-sectional shape, preferably a round cross-sectional shape. In further preferred embodiments, all pillar-shaped built-in elements of the secondary structure have the same (cross-sectional) diameter as well as the same height. In further preferred embodiments, as outlined above, the spacings between the pillar-shaped built-in elements of the secondary structure should be greater than the smallest spacings of the structures which form the preferably zigzag-shaped filter structure of the main filter located downstream of secondary structure within the fluid distribution area.

In advantageous embodiments, the plurality of pillar-shaped built-in elements may be arranged in a plurality of parallel rows, arranged transversally to the direction of flow and preferably extending from one sidewall of the base plate to the opposite sidewall. Furthermore, in particular embodiments, the plurality of pillar-shaped built-in elements are arranged in about 40 to about 70 parallel rows per mm (with regard to the length of the secondary structure in the direction of flow), preferably in about 50 to 60 parallel rows per mm, extending from one sidewall to the opposite sidewall.

In yet further embodiments, the plurality of pillar-shaped built-in elements of the secondary structure may be arranged in about 10 to about 30, preferably in about 15 to about 25 parallel rows extending from one sidewall to the opposite sidewall perpendicular to the direction of flow. In further embodiments, each row of pillar-shaped built-in elements of the secondary structure may comprise from about 40 to about 60, preferably from about 45 to about 55 built-in elements per mm. In specific embodiments, each row of pillar-shaped built-in elements of the secondary structure may comprise from about 80 to about 120, preferably from about 90 to about 110 built-in elements per row.

In these embodiments, especially in cases in which the rows are arranged relatively to another in an ABAB arrangement as described above, high densities of pillar-shaped built-in elements can be realized in the secondary structure located within the fluid distribution area ranging from about 200,000 (two hundred thousand) to about 300,000 or from about 250,000 to about 300,000 built-in elements per cm² (with regard to the surface of the fluid distribution area).

Accordingly, in preferred embodiments, the built-in elements are provided in the fluid distribution area in a number of from about 200,000 to about 300,000 per cm² or of from about 250,000 to about 300,000 per cm².

As already described in detail above, the fluid distribution area is arranged between the inlet and the main filter area in the direction of flow of the medically active fluid. In preferred embodiments, however, the fluid distribution area does not overlap with the main filter area, while, in specific embodiments, it may be possible that the liquid distribution area borders the man filter area. In other words, in some embodiments, the downstream end of the fluid distribution area may contact the upstream end of the main filter area. In further embodiments, however, the pillar shaped built-in elements of the fluid distribution area do not contact the main filter area, especially not the protrusions of the primary structure located within the main filter area.

The fluid distribution area of the present microstructured nozzle or, more specifically, the secondary structure comprising the arrangement of pillar-shaped built in elements provided therein, in preferred embodiments, allows for a more even and equal or, in other words, homogeneous distribution of the medically active fluid to be filtered and atomized over a broader segment or even of the entire width of the main filter, especially in cases in which the medically active fluid does not evenly enter the inlet of the microstructured nozzle over the entire width of the fluid channel extending from one sidewall to the opposite sidewall. This may be especially advantageous in cases in which the pressurized medically active fluid is delivered to the inlet of the microstructured nozzle from a pumping unit or other source of pressure via a tubing or other fluidic connection which has a cross-sectional diameter smaller than the width of the inlet of the microstructured nozzle. In these cases, the pressurized medically active fluid enters the secondary structure of the fluid distribution area at its upstream end (facing the inlet of the microstructured nozzle) and fills the fluid channels located between the pillar-shaped built-in elements before passing through the filter channels of primary structure of the main filter, especially in cases in which, according to preferred embodiments of the present microstructured nozzle, the spacings between the built-in elements of the secondary structure are such that a resulting cross sectional area transverse to the direction of flow which is effectively permeable to the medically active fluid is greater than a corresponding effective cross sectional surface area of the main filter channels formed by the projections of the main filter.

Furthermore, in preferred embodiments, the secondary structure provided in the fluid distribution area, especially when provided in the form of parallel rows in an ABAB arrangement with preferably equidistant intervals within rows A and B and between rows A and B as described in detail above, allows for the formation of a fluid-gas interface, preferably at the most downstream row of pillar-shaped built-in elements, in cases in which the medically active fluid to be atomized moves or retracts in an upstream direction, i.e. from the main filter area towards the inlet of the microstructured nozzle. This may become relevant in cases in which the pressure exerted on the medically active fluid is (partly) relieved or even reversed, for example, after the ejection and atomization of the pressurized medically active fluid through the at least one ejection channel. Furthermore, this may become relevant in cases in which medically active fluid remaining in the microstructured nozzle is exposed to a negative pressure or, in other words, to an underpressure, for example to an underpressure generated by a pumping unit comprising a pumping chamber and a piston reciprocally moveable therein, during the priming phase of the pump. In these cases, the secondary structure comprising an array of pillar-shaped built-in elements may function as a microfluidic valve avoiding or reducing the backflow of medically active liquid in the upstream direction according to the capillary forces acting between the medically active liquid and the array of pillar-shaped built-in elements at the fluid-gas interface at the downstream end of the fluid distribution area.

In preferred embodiments, the secondary structure located within the liquid distribution area is provided in a form in which the forces acting between the medially active fluid and the array of pillar-shaped built-in elements are defined and, to the extent possible, uniform, independent of the actual location of the boundary line between the medically active fluid and the surrounding atmosphere. In cases in which the array of pillar-shaped built-in elements is provided in the form of equidistant parallel rows, this is especially the case when the boundary line is located at the most downstream row of built-in elements. In cases, however, in which a plurality of pillar-shaped built-in elements with uniform dimensions are evenly distributed over the entire fluid distribution area, the forces acting between the medically active fluid and the pillar structures at the boundary line (corresponding to the Laplace pressure of the medically active fluid contacting the plurality of pillar-shaped built-in elements) may be uniform independent of the actual location of the boundary line (assuming that the medically active fluid will form a linear or almost linear boundary line or meniscus perpendicular to the direction of flow between the opposing sides of the liquid channel). Accordingly, in preferred embodiments as already outlined above, the secondary structure is provided in the form of an array of uniform pillar-shaped built-in elements having a cylindrical circumferential wall which are arranged in equidistant parallel rows, preferably in an ABAB configuration, as described in detail above.

As outlined above, it may be advantageous if the forces acting between the medically active fluid and the array of pillar-shaped built-in elements are defined and, to the extent possible, uniform, independent of the actual location of the boundary line (corresponding to the fluid-gas interface) between the medically active fluid and the surrounding atmosphere, especially in cases in which the secondary structure of pillar-shaped built-in elements acts as a microfluidic valve or break, modulating the backflow of medically active liquid in the upstream direction as described above. In order to provide for uniform and defined forces acting on the medically active fluid and the array of pillar-shaped boundary elements it may be advantageous when the width of the fluid distribution area, specifically the width of the array of pillar-shaped boundary elements provided in the fluid distribution area is constant or substantially constant over the entire length of the fluid distribution area. Furthermore, in advantageous embodiments, the cross-sectional area (perpendicular to the direction of flow) of the fluid distribution area or, more specifically, the part of the flow channel spanning the fluid distribution area is constant or substantially constant over the entire length of the fluid distribution area. In cases in which the height of the flow channel is constant or substantially constant this, however, may be achieved by a constant width over the entire length of the flow channel.

Furthermore, it has been found that the positioning of the array of pillar-shaped built in elements of the secondary structure in an area upstream of the main filter area, or in other words, on the high pressure side of the main filter area on which the pressurized medically active fluid hits the main filter, may be advantageous as it provides for the further stabilization of the connection between the base plate and the cover plate of the present microstructured nozzle. This may be especially beneficial to reduce or prevent potential deformation of the base plate and/or the cover plate by the highly pressurized medically active fluid (of up to 300 bar or even higher) which would seriously affect the positioning of the microstructures provided on the base plate as well as their connection to the cover plate and, accordingly, could seriously affect the tightness of the present microstructured nozzle or the flow channels provided therein.

In further particular embodiments, the present microstructured nozzle may additionally comprise a first interface area located between the (outlet or downstream end of the) fluid distribution area and the (inlet or upstream end of the) main filter area in the direction of flow, wherein the first interface area does not comprise structural elements located within the first interface area, thereby supporting the formation of a fluid-gas interface at the outlet side of the distribution area.

In particular embodiments, such first interface area may be a hollow space located between the fluid distribution area which, in some embodiments, has a width and height essentially corresponding to the width and height of the (downstream end of the) fluid distribution area located upstream of the first interface area as well as to the width and height of the (upstream end of the) main filter area located downstream of the first interface area, as both described in detail above. In alternative embodiments, however, the first interface area may also have different dimensions, for example a narrower width than one or both of the neighboring areas while the height is the same as that of the neighboring areas. In some embodiments, the first interface area extends over the entire width of the flow channel from one opposing sidewall to the other opposing sidewall and, accordingly, may have a width in the range of up to about 5 mm, such as from about 0.5 to about 4 mm or from about 1 mm to about 4 mm or from about 1.5 mm to about 2.5 mm or from about 2 to about 3 mm. In further embodiments, the length of the first interface area or, in other words, the distance by which it spaces the main filter area and the liquid distribution area may be varied within broad ranges and may, for example, be selected within the range of from about 0.01 mm to about 0.5 mm, or from about 0.01 mm to about 0.1 mm or to about 0.05 mm.

In further particular embodiments, the present microstructured nozzle may additionally comprise a second interface area located between the inlet (of the present microstructured nozzle and the (inlet or upstream end of the) fluid distribution area, wherein the second interface area as well does not comprise structural elements located within the second interface area, thereby supporting the formation of a fluid-gas interface at the inlet side of the fluid distribution area.

In particular embodiments, such second interface area may be a hollow space located between the inlet (of the present microstructured nozzle) and the (inlet or upstream end of the) fluid distribution area which, in some embodiments, has a width and height essentially corresponding to the width and height of the fluid distribution area located downstream of the second interface area as well as to the width and height of the inlet of the microstructured nozzle located upstream of the second interface area, as both described in detail above. In alternative embodiments, however, the second interface area may also have different dimensions, for example a narrower width than the inlet or the fluid distribution area while the height is the same as that of the inlet and the fluid distribution area. In some embodiments, the second interface area extends over the entire width of the flow channel from one opposing sidewall to the other opposing sidewall and, accordingly, may have a width in the range of up to about 5 mm, such as from about 0.5 to about 4 mm or from about 1 mm to about 4 mm or from about 1.5 mm to about 2.5 mm or from about 2 to about 3 mm. In further embodiments, the length of the second interface area or, in other words, the distance by which it spaces the inlet of the microstructured nozzle and the liquid distribution area may be varied within broad ranges and may, for example, be selected within the range of from about 0.01 mm to about 0.5 mm, or from about 0.01 mm to about 0.1 mm or to about 0.05 mm.

In yet further embodiments, the present microstructured nozzle may comprise a coarse filter area located between the inlet (of the present microstructured nozzle) and the (inlet or upstream end of the) fluid distribution area or (if present) the second interface area, the coarse filter area comprising a coarse filter constructed as a tertiary structure, with a plurality of coarse filter projections arranged side by side in at least one row, each being formed as an integral component of the base plate and projecting therefrom, the projections being spaced from one another by coarse filter channels which form a path for fluid through the nozzle from the inlet to the outlet, while the cover plate, if it is attached to the base plate, covers the coarse filter projections and the coarse filter channels.

According to these embodiments, the optional course filter area of the present microstructured nozzle comprises a coarse filter area comprising a coarse filter constructed as the tertiary structure. The coarse filter, if present, is preferably located and formed on the coarse filter area of the base plate as described in further detail below. The course filter may, in some embodiments, comprise a plurality of coarse filter projections arranged side by side in at least one row, preferably in one row, whereas preferably each coarse filter projection may constitute an integral component of the base plate and may project therefrom. Furthermore, the coarse filter projections, if present, may be spaced from another by coarse filter channels which form a plurality of paths for the fluid from the inlet to the outlet (via the second interface area (if present), the fluid distribution area, the first interface area (if present) and the main filter area), while the cover plate, if it is attached to the base plate, covers the coarse filter projections and the coarse filter channels.

The coarse filter area, in some embodiments, extends over the entire width of the flow channel from one opposing sidewall to the other opposing sidewall and, accordingly may have a width in the range of up to about 5 mm, such as from about 0.5 to about 4 mm or from about 1 mm to about 4 mm or from about 1.5 mm to about 2.5 mm or from about 2 to 3 mm and a length (in the direction of flow) of from about 0.05 mm to about 0.5 mm or from about 0.1 mm to about 0.3 mm or to about 0.2 mm.

In particular embodiments, in the microstructured nozzle according to this aspect of the invention, the coarse filter comprises a plurality of projections extending transversely to the flow direction from the base plate, defining a plurality of coarse filter channels. In preferred embodiments, the coarse filter projections may have a rectangular or square cross-sectional shape, however other shapes such as circular, oval or irregular shapes are also possible. In further particular embodiments, the projections of the coarse filter are arranged side by side over an entire width of the coarse filter area or, in other words, from one sidewall of the base plate to the opposing sidewall of the base plate. In some embodiments, the coarse filter projections may have a width perpendicular to the direction of flow of from about 0.01 mm to about 0.1 mm, or from about 0.025 mm to about 0.075 mm and a length (in the direction of flow) of from about 0.05 mm to about 0.3 mm, or from about 0.1 mm to about 0.3 mm or to about 0.2 mm. In further embodiments, the coarse filter projections may be evenly distributed over the entire width of the flow channel, preferably at a density of from about 3 to about 7 projections per centimeter. In further embodiments, the coarse filter channels may be provided with a width of from about 0.05 mm to about 0.3 mm, or from about 0.1 mm to about 0.2 mm.

As described in detail above, the present microstructured nozzle comprises at least an inlet, an outlet and, located between the inlet and the outlet in the direction of flow,
a fluid distribution area comprising a secondary structure of pillar-shaped built-in elements located downstream of the inlet,
a main filter area comprising a main filter as the primary structure located downstream of the fluid distribution area, and
a filtrate outlet area located downstream of the main filter area.

In further embodiments, the present microstructured nozzle comprises at least an inlet, an outlet and, located between the inlet and the outlet in the direction of flow,
a fluid distribution area comprising a secondary structure of pillar-shaped built-in elements located downstream of the inlet,
a first interface area located downstream of the fluid distribution area,
a main filter area comprising a main filter as the primary structure located downstream of the first interface area, and
a filtrate outlet area located downstream of the main filter area.

In further embodiments, the present microstructured nozzle comprises at least an inlet, an outlet and, located between the inlet and the outlet in the direction of flow,
a second interface area located downstream of the inlet,
a fluid distribution area comprising a secondary structure of pillar-shaped built-in elements located downstream of the second interface area,
a first interface area located downstream of the fluid distribution area,
a main filter area comprising a main filter as the primary structure located downstream of the first interface area, and
a filtrate outlet area located downstream of the main filter area.

In yet further embodiments, the present microstructured nozzle comprises at least an inlet, an outlet and, located between the inlet and the outlet in the direction of flow,
a coarse filter area comprising a coarse filter as a tertiary structure located downstream of the inlet,
a second interface area located downstream of the coarse filter area,
a fluid distribution area comprising a secondary structure of pillar-shaped built-in elements located downstream of the second interface area,
a first interface area located downstream of the fluid distribution area,
a main filter area comprising a main filter as the primary structure located downstream of the first interface area, and
a filtrate outlet area located downstream of the main filter area.

As already mentioned above, the present microstructured nozzle may be comprised by an inhalation device or, in other words, nebulizer or atomizer for inhalation therapy. Accordingly, in a second aspect, the present invention provides for an inhalation device for inhalation therapy which comprises a microstructured nozzle according to the first aspect of the invention. For the avoidance of doubt, it should be noted that all features, embodiments, explanations or combinations thereof as described in detail above in connection with the microstructured nozzle of the first aspect of the invention equally apply to the inhalation device of this second aspect of the invention, wherever applicable.

As already mentioned above, inhalation devices for the generation of an inhalable aerosol of a medically active fluid have been described in the prior art, for example in US 2005/0001076 A1 as discussed above and the references cited therein. Another inhalation device has been disclosed in WO 2018/197730 A1 the contents of which are herewith incorporated by reference in their entirety. These inhalation devices usually have a small size so that they can be held and operated by a user with a single hand. They usually comprise a reservoir for holding the medically active fluid to be aerosolized and administered, a pumping unit for pressurizing the medically active liquid, specifically a pumping unit adapted for the generation of discontinuous, defined volumes of highly pressurized medically active fluid, an actuating mechanism as well as a nozzle unit through which the medically active fluid pressurized by the pumping unit is aerosolized or, in other words, atomized. The defined volumes of medically active fluid to be aerosolized may be selected within a broad range, in some embodiments, within a range of from about 1 μL to about 50 μL or of from about 10 μL to about 25 μL, such as about 15 μL. According to this second aspect of the present invention, the present inhalation device comprises a microstructured nozzle according to the first aspect of the invention as described in detail above. Due to the advantages of the microstructured nozzle according to the first aspect of the invention as described in detail above, the inhalation device according to this second aspect of the invention allows for a simplified design of such inhalation device as well as for a longer operating live thereof due to the advantageous characteristics of the microstructured nozzle interacting with the further functional units of the present inhalation device. Such advantageous characteristics may comprise but not be limited to enhanced mechanical robustness of the as well as advantageous filtration properties which, inter alia, allows for the simplified design of the further units of the inhalation device, for example the pumping unit.

Amongst others, the present invention relates to the following specific embodiments:
1. Microstructured nozzle (1) for a device (100) for the generation of an inhalable aerosol of a medically active fluid (2), the microstructured nozzle having a main filter (21), an inlet (3) for unfiltered fluid and an outlet (4) for filtered fluid, the inlet and the outlet defining a direction of flow (X) of the fluid from the inlet to the outlet, the nozzle comprising:
   a substantially flat base plate (5) and a cover plate (6) which may be attached thereto;
   a main filter area (20) comprising the main filter (21) constructed as the primary structure, with a plurality of main filter projections (22) arranged side by side in at least one row (23), each main filter projection being formed as an integral component of the base plate and projecting therefrom, the main filter projections being spaced from one another by main filter channels (24) which form a path for the fluid through the nozzle from the inlet to the outlet, while the cover plate, if it is attached to the base plate, covers the main filter projections and the main filter channels;
   a filtrate outlet area (30) arranged between the main filter and the outlet in the direction of flow; and
   a fluid distribution area (40) arranged between the inlet and the main filter area in the direction of flow (for distribution of the unfiltered fluid prior to contact with the main filter),
   wherein in the fluid distribution area is disposed a secondary structure (41) which comprises a plurality of pillar-shaped built-in elements (42) extending from the base plate and/or the cover plate transversely to the flow direction.
2. Microstructured nozzle according to item 1, wherein the inlet is located at the inlet end (7) of the base plate and the outlet is located at the opposite outlet end (8) of the base plate, and wherein the inlet and the outlet are connected by opposing sidewalls (9, 10) the inlet and outlet defining a flow channel (11) through which the medically active fluid flows in the direction of flow (X), wherein the outlet comprises at least one ejection channel (12) for the ejection of a jet of the medically active fluid.
3. Microstructured nozzle (1) according to item 2, wherein the projections (22) of the main filter (21) are arranged side from one sidewall (9) of the base plate (5) to the opposing sidewall (10) of the base plate (5).
4. Microstructured nozzle according to any one of the preceding items, wherein the outlet comprises at least two ejection channels adapted to eject at least two jets of the medically active fluid such that the at least two jets intersect with each other to form the inhalable aerosol.
5. Microstructured nozzle according to any one of the preceding items, wherein the main filter comprises a plurality of zigzag projections extending transversely to the flow direction from a base plate, defining a plurality of channels (24) and forming spikes (25) in directions of the inlet and the outlet.
6. Microstructured nozzle according to any one of the preceding items, wherein the filtrate outlet area does not comprise structural elements located in the inner volume of the filtrate outlet area.
7. Microstructured nozzle according to any one of the preceding items, wherein one or more spacings between the built-in elements (of the secondary structure), each of which forms a throughflow channel for the liquid passing through, are such that a resulting cross sectional area transverse to the direction of flow which is effectively permeable to the liquid is greater than a corresponding effective cross sectional surface area of the main filter channels formed by the projections of the main filter such that the built-in elements do not substantially increase a flow resistance.
8. Microstructured nozzle according to any one of the preceding items, wherein the built-in elements of the fluid distribution area have a cylindrical circumferential wall.
9. Microstructured nozzle according to any one of the preceding items, wherein the built-in elements are at a spacing of from about 0.005 mm to about 0.02 mm from one another.
10. Microstructured nozzle according to any one of the preceding items, wherein the built-in elements have a diameter of from about 0.005 mm to about 0.02 mm.
11. Microstructured nozzle according to any one of the preceding items, wherein the pillar-shaped built-in elements extend from the base plate to the cover plate.
12. Microstructured nozzle according to any one of the preceding items, wherein the projections of the main filter are arranged side by side over an entire width of the filter.
13. Microstructured nozzle according to any one of the preceding items, wherein the built-in elements are formed as an integral part of the base plate.
14. Microstructured nozzle according to any one of the preceding items, wherein all of the main filter projections and all of the built-in elements are formed as an integral part of the base plate.
15. Microstructured nozzle according to any one of the preceding items, wherein the plurality of pillar-shaped built-in elements are arranged in a plurality of parallel rows (33), arranged transversally to the direction of flow.
16. Microstructured nozzle according to any one of the preceding items, wherein the plurality of pillar-shaped built-in elements are arranged in about 40 to 70 parallel rows per mm (with regard to the length of the secondary structure in the direction of flow), preferably in about 50 to 60 parallel rows per mm, extending from one sidewall to the opposite sidewall.
17. Microstructured nozzle according to any one of the preceding items, wherein the plurality of pillar-shaped built-in elements are arranged in about 10 to about 30, preferably in about 15 to about 25 parallel rows extending from one sidewall to the opposite sidewall perpendicular to the direction of flow.
18. Microstructured nozzle according to any one of the preceding items, wherein each row of pillar-shaped built-in elements of the secondary structure comprises about 40 to about 60, preferably from about 45 to about 55 built-in elements per mm.
19. Microstructured nozzle according to any one of the preceding items, wherein each row of pillar-shaped built-in elements of the secondary structure comprises about 80 to about 120, preferably from about 90 to about 110 built-in elements per row.
20. Microstructured nozzle according to any one of the preceding items, wherein at least some built-in elements form equilateral hexagonal designs, where a center of each of the hexagonal designs is formed by a built-in element and each angle of each of the hexagonal designs is formed by adjacent built-in elements.
21. Microstructured nozzle according to any one of the preceding items, wherein the built-in elements are provided in the fluid distribution area in a number of about 200,000 to about 300,000 per cm².
22. Microstructured nozzle according to any one of the preceding items, wherein the fluid distribution area does not overlap with the main filter area.
23. Microstructured nozzle according to any one of the preceding items, wherein the built-in elements (of the secondary structure) of the fluid distribution area do not contact the main filter area.
24. Microstructured nozzle according to any one of the preceding items, wherein the width of the fluid distribution area, specifically the width of the array of pillar-shaped boundary elements provided in the fluid distribution area is constant or substantially constant over the entire length of the fluid distribution area.
25. Microstructured nozzle according to any one of the preceding items, comprising a first interface area (50) located between the fluid distribution area and the main filter area in the direction of flow, wherein the first interface area does not comprise structural elements located within the first interface area.
26. Microstructured nozzle according to any one of the preceding items, comprising a second interface area (60) located between the inlet and the fluid distribution area in the direction of flow, wherein the second interface does not comprise structural elements located within the second interface area.
27. Microstructured nozzle according to any one of the preceding items, comprising a coarse filter area (70) located between the inlet and the fluid distribution area or the second interface area, the coarse filter area comprising a coarse filter (71) constructed as the tertiary structure, with a plurality of coarse filter projections (72) arranged side by side in at least one row, each being formed as an integral component of the base plate and projecting therefrom, the projections being spaced from one another by coarse filter channels (73) which form a path for fluid through the nozzle from the inlet to the outlet, while the cover plate, if it is attached to the base plate, covers the coarse filter projections and the coarse filter channels.
28. Inhalation device (100) for inhalation therapy which comprises a microstructured nozzle according to any one of items 1 to 27.

### DETAILED DESCRIPTION OF THE DRAWINGS

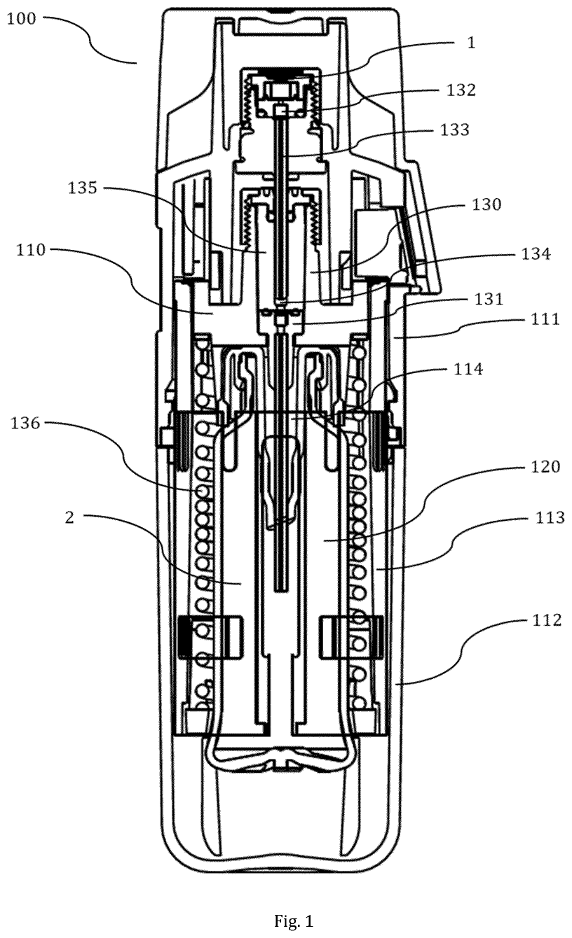

FIG. 1 shows an inhalation device (100) comprising an inhalation device unit (110) and an exchangeable reservoir (120) in the form of a cartridge inserted into the inhalation device (100) and containing the medically active fluid (2). The inhalation device unit (110) has a housing (111) with a lower part (112) that can be detached from the inhalation device unit (110) and removed to open the housing (111) and allow access to the receiving unit (113) in which the exchangeable reservoir in the form of a cartridge (120) can be inserted. The receiving unit (113) further has a connection unit (114) adapted to releasably and fluidically connect to a connection port of the exchangeable reservoir (120).

The inhalation device unit (110) further has a microstructured nozzle (1) located at the downstream end of the inhalation device unit (110) for nebulization of the medically active fluid (2). The inhalation device (100) further has a pumping unit (130) which is arranged within the housing (111). As described in detail above, the pumping unit (130) is fluidically connected to the reservoir (120) (via the connection unit (114) of the receiving unit (113)) and to the nozzle (1) and is adapted to pump the medically active fluid (2) in a downstream direction from the reservoir (120) to the nozzle (1).

The pumping unit (130) has an upstream end (131) that is fluidically connected to the exchangeable reservoir (120), a downstream end (132) that is fluidically connected to the nozzle (1), wherein the pumping unit (130) further comprises (i) a riser pipe (133) having an upstream end (134), wherein the riser pipe (133) is adapted to function as a piston in the pumping unit (130), and wherein the riser pipe (133) is firmly affixed to the user-facing (downstream) side of the housing (111) such as to be immobile relative to the housing (111), and (ii) a hollow cylinder (135) located upstream of the riser pipe (133), wherein the upstream end of the riser pipe (134) is inserted in the cylinder (135) such that the cylinder (135) is longitudinally movable on the riser pipe (133).

As also shown in FIG. 1, the pumping unit (130) comprises (iii) a lockable means for storing potential energy (136) when locked and for releasing the stored energy when unlocked, the means (136) being arranged outside of, and mechanically coupled to, the cylinder (135) such that unlocking the means (136) results in a propulsive longitudinal movement of the cylinder (135) towards the downstream end of the pumping unit (132) and, thereby, resulting in the ejection of the pressurized medically active fluid (2) through the microstructured nozzle (1).

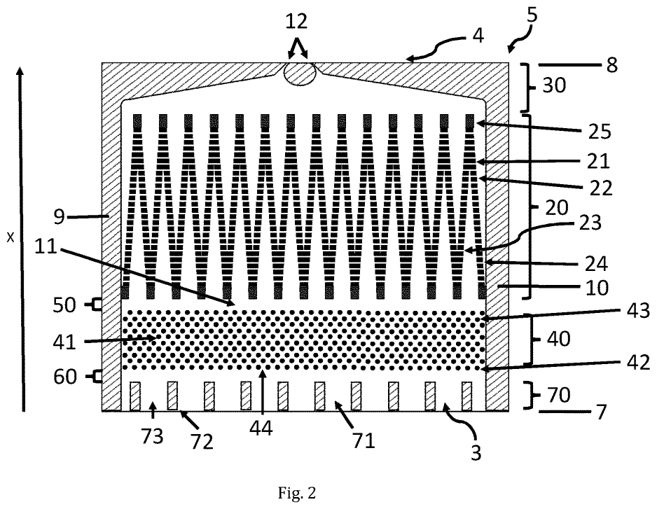

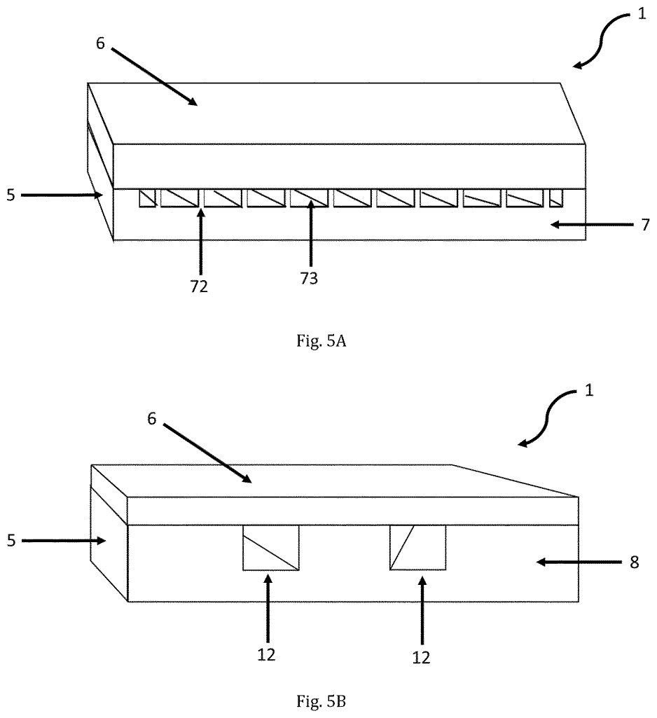

FIG. 2 depicts an embodiment of the base plate (5) of the present microfluidic nozzle (1), viewed from the side which is initially open and which may be subsequently covered with a cover plate (6) (see FIG. 5A/B). The microfluidic nozzle (1) has an inlet (3) located on the inlet end (7) of the base plate as well as an outlet (4) located on the outlet end (8) of the base plate (5), respectively, whereas the inlet (3, 7) and the outlet (4, 8) define a direction of flow (X) of the medically active fluid in a downstream direction from the inlet (3,7) towards the outlet (4,8). The outlet (4) comprises two ejection channels (12) through which jets of the medically active fluid may be ejected.

As can be seen in FIG. 2, base plate (5) comprises a main filter area (20) comprising the main filter (21) constructed as the primary structure. The main filter (21) comprises a plurality of main filter projections (22) arranged side by side in at least one row (23) (as shown in more detail in FIG. 4) folded in a zigzag-like structure forming spikes (25) and extending from one sidewall (9) to the opposite sidewall (10), thereby extending over the entire width of flow channel (11). Each main filter projection (22) is formed as an integral component of the base plate and projecting therefrom (perpendicular to the plane of projection). As also shown in the enlarged detail if FIG. 4, the main filter projections (22) being spaced from one another by main filter channels (24) which form a path for the fluid through the nozzle from the inlet (3, 7) to the outlet (4, 8). The inlet (3, 7) and the outlet (4, 8) are connected by opposing sidewalls (9, 10) of the base plate (5) thereby defining a flow channel (11) through which the medically active fluid flows from the inlet (3,7) in the downstream direction to the outlet (4,8), more specifically to the ejection channels (12).

Base plate (5) further comprises filtrate outlet area (30) arranged between the main filter area (20) or the main filter (21) and outlet (4, 8) with ejection channels (12) in the direction of flow. As can be seen in the embodiment of FIG. 2, filtrate outlet area (30) is a hollow space or volume with no structural elements provided therein connecting the downstream end of main filter area (20) with outlet (4, 8) and ejection channels (12).

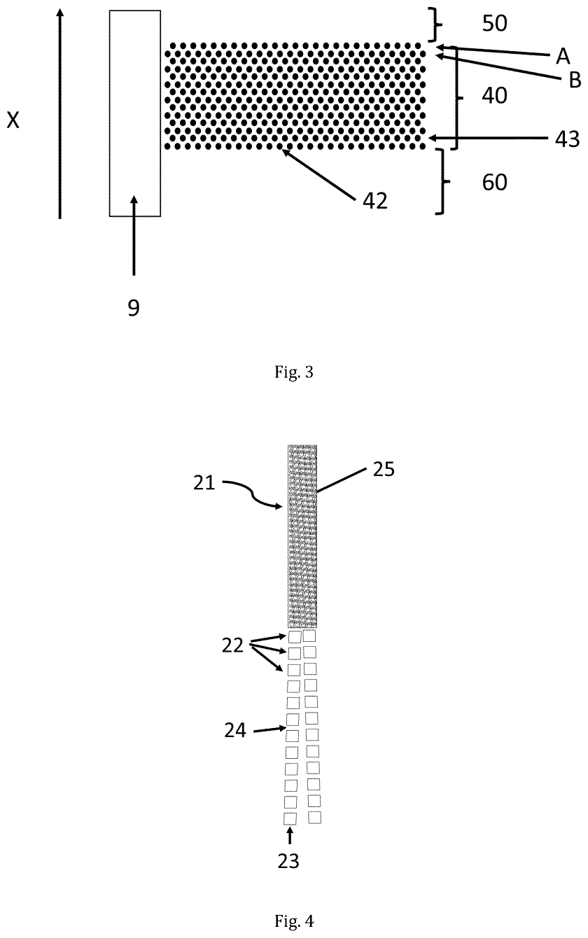

Furthermore, base plate (5) as shown in the embodiment of FIG. 2 comprises a fluid distribution area (40) which is arranged between the inlet (3, 7) and the main filter area (20) in the direction of flow. In the fluid distribution area (40) is disposed a secondary structure (41) in form of an array of a plurality of evenly spaced pillar-shaped built-in elements (42) extending from base plate (5). In FIG. 2, due to the top view on the plurality of pillar-shaped built-in elements (42) of the secondary structure (41), these are shown as circles corresponding to the top surfaces of the plurality of cylindrical pillars (42) having a circular cross section. As can be best seen in the enlarged detail of FIG. 3, the plurality of pillar-shaped built-in elements (42) as shown in this embodiment are arranged in a plurality of parallel rows (43), arranged transversally to the direction of flow (X). Furthermore, in the particular embodiment shown, the rows (43) of pillar-shaped built-in elements (42) are arranged in “ABAB” arrangement with preferably equidistant intervals within rows A and B and between rows A and B such that built-in elements (42) which are not located adjacent to the upstream or downstream end of the fluid distribution area (40) or to the opposing sidewalls (9, 10) form equilateral hexagonal designs, where a center of each of the hexagonal designs is formed by a built-in element (42) and each angle of each of the hexagonal designs is formed by adjacent built-in elements (42). The pillar-shaped built-in elements (42) of the secondary structure (41) are spaced from each other by channels (44) of the secondary structure.

As can be also seen in FIG. 2, the fluid distribution area (40) does not overlap with the main filter area (20) and, more specifically, pillar-shaped built-in elements (42) located in fluid distribution area (40) do not contact the main filter area (20).

Further, in the embodiment as shown in FIG. 2 fluid distribution area (40) which is evenly equipped with the array of pillar-shaped built-in elements (42). Furthermore, the width of the fluid distribution area (40) perpendicular to the direction of flow (X) as well as the width of the array of pillar-shaped built-in elements (42) provided in the fluid distribution area (40) is constant or substantially constant over the entire length of the fluid distribution area (40) and extends from one of the opposing sidewalls (9) to the other one (10).

Base plate (5) as shown in FIG. 2 further comprises a first interface area (50) located between fluid distribution area (40) and main filter area (20) in the direction of flow (X). As can be seen in this embodiment, the first interface area (50) does not comprise structural elements located within the first interface area (50) resulting in a hollow space or volume spacing and connecting the downstream end of fluid distribution area (40) from/with the upstream end of main filter area (20) and the main filter (21) located therein.

Furthermore, base plate (5) as shown in FIG. 2 further comprises a second interface area (60) located between inlet (3, 7) and fluid distribution area (40) in the direction of flow (X). As can be seen in this embodiment, the second interface area (60) does also not comprise structural elements located within the second interface area (60) resulting in a hollow space or volume spacing and connecting inlet (3, 7) or, more specifically, coarse filter area (70) as will be described below, from/with the upstream end of fluid distribution area (40) and the pillar-shaped built-in elements (42) located therein.

Base plate (5) according to the embodiment as shown in FIG. 2 further comprises a coarse filter area (70) located between the inlet (3, 7) and fluid distribution area (40) or, more specifically between the inlet (3,7) and the second interface area (60) in the direction of flow (X). The coarse filter area (70) comprises a coarse filter (71) constructed as the tertiary structure, with a plurality of coarse filter projections (72). In the embodiment shown in FIG. 2, the coarse filter projections (72) are provided in the form of rectangular structures which, as the projections (22) of the main filter (21) and the pillar-shaped built-in elements (42) of the secondary structure are integrally formed with base plate (5) and project perpendicular to the plane of projection from base plate (5). In the embodiment shown in FIG. 2, the coarse filter projections (72) are arranged side by side in one row of equally sized and shaped projections, each being formed as an integral component of the base plate and projecting therefrom. The coarse filter projections (72) are spaced from one another by coarse filter channels (73) which form a path for the medically active fluid through the nozzle from the inlet (3, 7) to the outlet (4, 8), while the cover plate (6, see FIG. 5A/B) if it is attached to base plate (5), covers the coarse filter projections (72) and the coarse filter channels (73).

As also apparent from FIG. 2, the width of the coarse filter channels (73) perpendicular to the direction of flow (X) is larger than the width of the channels (44) between the pillar-shaped built-in elements (42), in some embodiments by an order of magnitude, to filter coarse physical impurities or debris potentially comprised by the medically active fluid before it contacts the secondary structure of fluid distribution area (40) or the main filter (21).

FIG. 3 shows an enlarged top-view of the pillar-shaped built-in elements (42) of secondary structure as provided in fluid distribution area (40), specifically an enlarged detail of the portion of the secondary structure adjacent to left sidewall (9) as well as adjacent to the first interface area (50) located downstream as well as adjacent to the second interface area (60) located upstream of the fluid distribution area (40). In the embodiment as shown in FIG. 3, the pillar-shaped built-in elements (42) of the secondary structure are provided in the form of an array comprising equidistant rows (43) of a plurality of evenly spaced built-in elements (42) in an ABAB configuration as described in detail above. In this embodiment, the top or most downstream row of pillar-shaped built-in elements corresponds to row A wherein the second most downstream row correspond to row B, followed by a further row A and so forth. Most notably, due to this arrangement only two different distances between sidewall (9) and the corresponding sets of rows exits, resulting in the formation of defined capillary forces acting between the sidewall, the pillar-shaped built-in elements (42) and the medically active fluid.

FIG. 4 shows an enlarged top-view of a section of the main filter (21) located in the main filter area (20) with main filter projections (22) arranged in one row (23) and separated by main filter channels (24). More specifically, FIG. 5 shows a spike (25) of the zigzag-formed main filter (21) as shown in FIG. 2.

FIGS. 5A and 5B show perspective views of a microstructured nozzle (1) according to the present invention comprising base plate (5) and cover plate (6) attached to each other, thereby forming the fully assembled microstructured nozzle (1). More specifically, FIG. 5A shows the upstream end (7) of assembled microstructured nozzle (1) with inlet (3) and coarse filter projections (72) as well as coarse filter channels (73) whereas FIG. 5B shows the opposite downstream end (8) of assembled microstructured nozzle (1) with two ejection channels (12).

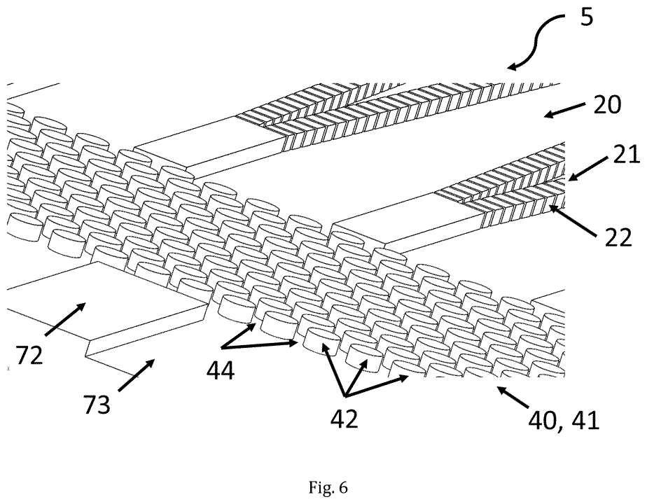

FIG. 6, finally, shows a perspective view of a section of base plate (5) of microstructured nozzle (1) as shown in top-view in FIG. 2. The section shown comprises portions of the main filter (21) located in the main filter area (20), the secondary structure (41) of fluid distribution area (40) with pillar-shaped built-in elements (42) spaced by channels (44) of the secondary structure and a coarse filter projection (72) with coarse filter channel (73). Due to the perspective projection, the height of the primary, secondary and tertiary structures, namely the height of the main filter projections (22), of the pillar-shaped built-in elements (42) as well as of the coarse filter projections (73) emerging perpendicular from base plate (5) can be seen.

### LIST OF REFERENCES

1 Microstructured nozzle
2 Medically active fluid
3 Inlet (of microstructured nozzle)
4 Outlet (of microstructured nozzle)
5 Base plate
6 Cover plate
7 Inlet end (of base plate)
8 Outlet end (of base pate)
9,10 Opposing sidewalls (of base plate)
11 Flow channel (of microstructured nozzle)
12 Ejection channel
20 Main filter area
21 Main filter
22 Main filter projections
23 Rows of main filter projections
24 Main filter channels
25 Spikes (of main filter)
30 Filtrate outlet area
40 fluid distribution area
41 secondary structures (of fluid distribution area)
42 Pillar-shaped built-in elements (of secondary structure)
43 Rows of built-in elements
44 Channels of the secondary structure
50 First interface area
60 Second interface area
70 Coarse filter area
71 Coarse filter
72 Coarse filter projections
73 Coarse filter channels
100 Inhalation device
110 Inhalation device unit
111 Housing
112 Lower part of housing
113 Receiving unit
114 Connection unit
120 Exchangeable reservoir
130 Pumping unit
131 Upstream end of pumping unit
132 Downstream end of pumping unit
133 Riser pipe
134 Upstream end of riser pipe
135 Hollow cylinder
136 Lockable means
X Direction of flow

1. Microstructured nozzle (1) for a device (100) for the generation of an inhalable aerosol of a medically active fluid (2), the microstructured nozzle (1) having a main filter (21), an inlet (3) for unfiltered fluid (2) and an outlet (4) for filtered fluid (2), the inlet (3) and the outlet (4) defining a direction of flow (X) of the fluid (2) from the inlet (3) to the outlet (4), the nozzle (1) comprising:
a substantially flat base plate (5) and a cover plate (6) which may be attached thereto;
a main filter area (20) comprising the main filter (21) constructed as the primary structure, with a plurality of main filter projections (22) arranged side by side in at least one row (23), each main filter projection (22) being formed as an integral component of the base plate (5) and projecting therefrom, the main filter projections (22) being spaced from one another by main filter channels (24) which form a path for the fluid (2) through the nozzle (1) from the inlet (2) to the outlet (3), while the cover plate (6), if it is attached to the base plate (5), covers the main filter projections (22) and the main filter channels (24);
a filtrate outlet area (30) arranged between the main filter (21) and the outlet (4) in the direction of flow (X); and
a fluid distribution area (40) arranged between the inlet (3) and the main filter area (20) in the direction of flow (X),
wherein in the fluid distribution area (40) is disposed a secondary structure (41) which comprises a plurality of pillar-shaped built-in elements (42) extending from the base plate (5) and/or the cover plate (6) transversely to the flow direction.

2. Microstructured nozzle (1) according to claim 1, wherein the main filter (21) comprises a plurality of zigzag projections (22) extending transversely to the flow direction from the base plate (5), defining a plurality of channels (24) and forming spikes (25) in directions of the inlet (3) and the outlet (4).

3. Microstructured nozzle (1) according to claim 1 or 2, wherein all of the pillar-shaped built-in elements (42) of the secondary structure provided in the present microstructured nozzle (1) are disposed in the fluid distribution area (40).

4. Microstructured nozzle (1) according to any one of the preceding claims, wherein the pillar-shaped built-in elements (42) of the secondary structure are evenly and regularly distributed over the entire fluid distribution area.

5. Microstructured nozzle (1) according to any one of the preceding claims, wherein the filtrate outlet area (30) does not comprise structural elements located in the inner volume of the filtrate outlet area (30).

6. Microstructured nozzle (1) according to any one of the preceding claims, wherein the filtrate outlet area (30) has the same width as the (downstream end) of the main filter area (20) and optionally gradually or discontinuously narrows in the direction towards the outlet end (4) of the microstructured nozzle (1).

7. Microstructured nozzle (1) according to any one of the preceding claims, wherein the inlet (3) is located at the inlet end (7) of the base plate (5) and the outlet (4) is located at the opposite outlet end (8) of the base plate (5), and wherein the inlet (3) and the outlet (4) are connected by opposing sidewalls (9, 10) the inlet (3) and outlet (4) defining a flow channel (11) through which the medically active fluid (2) flows in the direction of flow (X), wherein the outlet (4) comprises at least one ejection channel (12) for the ejection of a jet of the medically active fluid (2).

8. Microstructured nozzle (1) according to any one of the preceding claims, wherein the outlet (4) comprises at least two ejection channels (12) adapted to eject at least two jets of the medically active fluid (2) such that the at least two jets intersect with each other to form the inhalable aerosol.

9. Microstructured nozzle (1) according to any one of the preceding claims, wherein one or more spacings between the built-in elements (42) of the secondary structure, each of which forms a throughflow channel (44) for the liquid (2) passing through, are such that a resulting cross sectional area transverse to the direction of flow which is effectively permeable to the liquid (2) is greater than a corresponding effective cross sectional surface area of the main filter channels (24) formed by the projections (22) of the main filter (21) such that the built-in elements (42) do not substantially increase a flow resistance.

10. Microstructured nozzle (1) according to any one of the preceding claims, wherein the built-in elements (42) of the fluid distribution area have a cylindrical circumferential wall.

11. Microstructured nozzle (1) according to any one of the preceding claims, wherein the built-in elements (42) are at a spacing of from about 0.005 mm to about 0.02 mm from one another.

12. Microstructured nozzle (1) according to any one of the preceding claims, wherein the built-in elements (42) have a diameter of from about 0.005 mm to about 0.02 mm.

13. Microstructured nozzle (1) according to any one of the preceding claims, wherein the pillar-shaped built-in elements (42) extend from the base plate (5) to the cover plate (6).

14. Microstructured nozzle (1) according to any one of the preceding claims, wherein the projections (22) of the main filter (21) are arranged side by side over an entire width of the main filter (21).

15. Microstructured nozzle (1) according to any one of the preceding claims, wherein the built-in elements (42) are formed as an integral part of the base plate (5).

16. Microstructured nozzle (1) according to any one of the preceding claims, wherein all of the main filter projections (22) and all of the built-in elements (42) are formed as an integral part of the base plate (5).

17. Microstructured nozzle (1) according to any one of the preceding claims, wherein the plurality of pillar-shaped built-in elements (42) are arranged in a plurality of parallel rows (43), arranged transversally to the direction of flow.

18. Microstructured nozzle (1) according to any one of the preceding claims, wherein the plurality of pillar-shaped built-in elements (42) are arranged in about 40 to about 70 parallel rows (43) per mm (with regard to the length of the secondary structure in the direction of flow), preferably in about 50 to 60 parallel rows (43) per mm, extending from one sidewall (9) to the opposite sidewall (10).

19. Microstructured nozzle (1) according to any one of the preceding claims, wherein the plurality of pillar-shaped built-in elements (42) are arranged in about 10 to about 30, preferably in about 15 to about 25 parallel rows (43) extending from one sidewall (9) to the opposite sidewall (10) perpendicular to the direction of flow.

20. Microstructured nozzle (1) according to any one of the preceding claims, wherein each row (43) of pillar-shaped built-in elements (42) of the secondary structure (41) comprises about 40 to about 60, preferably from about 45 to about 55 built-in elements (42) per mm.

21. Microstructured nozzle (1) according to any one of the preceding claims, wherein each row (43) of pillar-shaped built-in elements (42) of the secondary structure (41) comprises about 80 to about 120, preferably from about 90 to about 110 built-in elements (42) per row.

22. Microstructured nozzle (1) according to any one of the preceding claims, wherein at least some built-in elements (42) form equilateral hexagonal designs, where a center of each of the hexagonal designs is formed by a built-in element (42) and each angle of each of the hexagonal designs is formed by adjacent built-in elements (42).

23. Microstructured nozzle (1) according to any one of the preceding claims, wherein the built-in elements (42) are provided in the fluid distribution area (40) in a number of about 200,000 to about 300,000 per cm².

24. Microstructured nozzle (1) according to any one of the preceding claims, wherein the fluid distribution area (40) does not overlap with the main filter area (20).

25. Microstructured nozzle (1) according to any one of the preceding claims, wherein the built-in elements (42) of the secondary structure (41) of the fluid distribution area (40) do not contact the main filter area (20).

26. Microstructured nozzle (1) according to any one of the preceding claims, wherein the width of the fluid distribution area (40), specifically the width of the array of pillar-shaped boundary elements (42) provided in the fluid distribution area (40) is constant or substantially constant over the entire length of the fluid distribution area (40).

27. Microstructured nozzle (1) according to any one of the preceding claims, comprising a first interface area (50) located between the fluid distribution area (40) and the main filter area (20) in the direction of flow, wherein the first interface area (50) does not comprise structural elements located within the first interface area (50).

28. Microstructured nozzle (1) according to any one of the preceding claims, comprising a second interface area (60) located between the inlet (3) and the fluid distribution area (40) in the direction of flow, wherein the second interface area (60) does not comprise structural elements located within the second interface area (60).

29. Microstructured nozzle (1) according to any one of the preceding claims, wherein the microstructured nozzle (1) comprises at least an inlet (3), an outlet (4) and, located between the inlet (3) and the outlet (4) in the direction of flow:
a fluid distribution area (40) comprising a secondary structure of pillar-shaped built-in elements (42) located downstream of the inlet (3),
a first interface area (50) located downstream of the fluid distribution area (40),
a main filter area (20) comprising a main filter (21) as the primary structure located downstream of the first interface area (50), and
a filtrate outlet area (30) located downstream of the main filter area (20).

30. Microstructured nozzle (1) according to any one of the preceding claims, comprising a coarse filter area (70) located between the inlet (3) and the fluid distribution area (40) or the second interface area (60), the coarse filter area (70) comprising a coarse filter (71) constructed as the tertiary structure, with a plurality of coarse filter projections (72) arranged side by side in at least one row, each being formed as an integral component of the base plate (5) and projecting therefrom, the projections (72) being spaced from one another by coarse filter channels (73) which form a path for fluid through the nozzle (1) from the inlet (3) to the outlet (4), while the cover plate (6), if it is attached to the base plate (5), covers the coarse filter projections (72) and the coarse filter channels (73).

31. Microstructured nozzle (1) according to any one of the preceding claims, wherein the microstructured nozzle (1) comprises at least an inlet (3), an outlet (4) and, located between the inlet (3) and the outlet (4) in the direction of flow:
a coarse filter area (70) comprising a coarse filter (71) as a tertiary structure located downstream of the inlet (3),
a second interface area (60) located downstream of the coarse filter area (70),
a fluid distribution area (40) comprising a secondary structure of pillar-shaped built-in elements (42) located downstream of the second interface area (60),
a first interface area (50) located downstream of the fluid distribution area (40),
a main filter area (20) comprising a main filter (21) as the primary structure located downstream of the first interface area (50), and
a filtrate outlet area (30) located downstream of the main filter area (20).

32. Inhalation device (100) for inhalation therapy which comprises a microstructured nozzle (1) according to any one of claims 1 to 31.

33. Inhalation device (100) according to claim 32, wherein the inhalation device (100) is a handheld inhalation device.
